(19) United States
(12) Patent Application Publication
(10) Pub. No.: US 2026/0263243 A1
(43) Pub. Date: Sep. 10, 2026

(54) HYDRAULIC DAMPENER FOR A PROSTHETIC DEVICE

(71) Applicant: Össur Iceland ehf, Reykjavík (IS)

(72) Inventors: Ragnar Sverrisson, Reykjavik (IS); Halldor Einarsson, Reykjavik (IS); Joseph Daniel Hill, Salt Lake City, UT (US)

(21) Appl. No.: 19/557,646

(22) Filed: Mar. 5, 2026

Related U.S. Application Data
(60) Provisional application No. 63/768,566, filed on Mar. 7, 2025.

(51) Int. Cl. A61F 2/64; A61F 2/50

(52) U.S. Cl. CPC A61F 2/64; A61F 2002/5006

(57) ABSTRACT

A hydraulic dampener includes a piston-cylinder arrangement in fluid communication with a valve assembly. The valve assembly includes a valve spool with one or more valve spool ports, which is rotatably housed in a valve sleeve having one or more valve sleeve ports. The outer surface of the valve spool includes a plurality of axial grooves disposed between the valve spool ports that capture hydraulic fluid as it flows into the valve assembly and generate a thin film of hydraulic fluid around the outer surface of the valve spool to aid the rotation of the valve spool within the valve sleeve. A hydraulic resistance of the hydraulic valve assembly varies approximately exponentially with a change in valve position caused by rotating the valve spool relative to the valve sleeve to vary an alignment between the one or more valve core ports and one or more valve sleeve ports.

### INCORPORATION BY REFERENCE TO ANY PRIORITY APPLICATIONS

Any and all applications for which foreign or domestic priority is claimed are identified in the Application Data Sheet as filed with the present application and are hereby incorporated by reference under 37 CFR. 1.37. For example, this application claims priority to U.S. Provisional Patent Application No. 63/768,566, titled “HYDRAULIC DAMPENER FOR A PROSTHETIC DEVICE”, filed on Mar. 7, 2025, the content of which is incorporated herein by reference in its entirety and for all purposes.

### BACKGROUND

### Field

The present disclosure relates generally to a hydraulic dampener and valve system for use in a prosthetic device, more specifically, a prosthetic knee.

### Description of the Related Art

Prosthetics are used to replace and restore the functionality of amputated natural body parts. Hydraulics have long been used to control the resistances of prosthetic devices, such as knees, feet, and ankles, via valves that are actuated to adjust hydraulic resistance of the prosthetic devices. However, existing hydraulic designs are deficient in that friction between components of the valves can inhibit actuation of the valves, requiring increased torque to actuate the valves.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices, and methods for a hydraulic valve system for a prosthetic device, for example a microprocessor-controlled prosthetic knee.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

Some aspects of the present disclosure relate to a hydraulic dampener for a prosthetic device, including a hydraulic cylinder and a valve system in fluid communication with the hydraulic cylinder. The valve system may be operable to allow flow of a hydraulic fluid to and from the hydraulic cylinder. The valve assembly may include a valve spool having one or more valve spool ports and a plurality of recessed axial grooves, and a valve sleeve with one or more valve sleeve ports surrounding the valve spool so that the valve spool is rotatable relative to the valve sleeve. A hydraulic resistance of the valve assembly varies approximately exponentially with a change in valve position of the valve assembly caused by rotating the valve spool relative to the valve sleeve to vary an alignment between the one or more valve spool ports and the one or more valve sleeve ports.

In some embodiments, the valve spool comprises a first cylindrical body, and a valve post, the valve post extending distally from the first cylindrical body, the one or more valve spool ports arranged circumferentially around the first cylindrical body. Each of the one or more valve spool ports defines a small axial channel through which hydraulic fluid can flow through the valve assembly. In some embodiments, wherein the one or more valve sleeve ports is a curved valve sleeve port comprising an oblong portion and an elongate portion. In some embodiments, the valve sleeve comprises a second cylindrical body, and the one or more valve sleeve ports are arranged circumferentially around the second cylindrical body. In some embodiments, the valve sleeve further comprises one or more axial grooves disposed on the second cylindrical body between the one or more valve sleeve ports.

Certain aspects of the present disclosure relate to a hydraulic valve assembly having a valve spool with one or more valve spool ports and a plurality of recessed axial grooves, and a valve sleeve with one or more valve sleeve ports surrounding the valve spool so that the valve spool is rotatable relative to the valve sleeve, the valve sleeve comprising one or more valve sleeve ports. The hydraulic resistance of the valve assembly may vary approximately exponentially with a change in valve position of the valve assembly caused by rotating the valve spool relative to the valve sleeve to vary an alignment between the one or more valve spool ports and one or more valve sleeve ports. In some embodiments, the one or more valve sleeve ports are a curved valve sleeve port comprising an oblong portion and an elongate portion. In some embodiments, the elongate portion extends at an angle (e.g., between 2° and 3°) relative to a plane transverse to an axis of the valve assembly. The plane may be perpendicular the axis of the valve assembly. In some embodiments, each of the one or more valve spool ports defines a small axial channel through which hydraulic fluid can flow through the hydraulic valve assembly. The one or more valve spool ports may be multiple valve spool ports, and the one or more valve sleeve ports may be multiple valve sleeve ports. The multiple valve spool ports may be configured to at least partially overlap with the multiple valve sleeve ports. An area of overlap between the multiple valve sleeve ports and the multiple valve spool ports may increase exponentially during rotation of the valve spool within the valve sleeve. In some embodiments, the valve spool may further include one or more radial grooves disposed around its circumference.

Some aspects of the present disclosure are related to a device for actuation of a prosthetic device including a hydraulic cylinder, a piston movably coupled to the hydraulic cylinder and configured to linearly move within the hydraulic cylinder, and a motorized valve system. In some embodiments, the prosthetic device is a prosthetic knee. The motorized valve system may include a motor and a valve assembly including a valve spool having one or more valve spool ports and a plurality of recessed axial grooves, and a valve sleeve with one or more valve sleeve ports surrounding the valve spool so that the valve spool is rotatable relative to the valve sleeve. The hydraulic resistance of the valve assembly may vary approximately exponentially with a change in valve position of the valve assembly caused by rotating the valve spool relative to the valve sleeve to vary an alignment between the one or more valve spool ports and one or more valve sleeve ports. In some embodiments, the valve spool includes a first cylindrical body, and a valve post, the valve post extending distally from the first cylindrical body, the one or more valve spool ports arranged circumferentially around the first cylindrical body. In some embodiments, the valve spool further includes one or more axial channels disposed on the first cylindrical body between the one or more valve spool ports. Each of the valve spool ports defines a small axial channel through which hydraulic fluid can flow through the hydraulic valve assembly. In some embodiments, the valve sleeve includes a second cylindrical body, the one or more valve sleeve ports arranged circumferentially around the second cylindrical body. In some embodiments, the one or more valve sleeve ports are curved valve sleeve ports having an oblong portion with a first height, and an elongate portion with a second height, and wherein the first height is greater than the second height. In some embodiments, the device may further include a coupler configured to operably couple the motor to a distal end of the valve assembly, and actuation of the motor causes a rotation of the valve spool within the valve sleeve. In some embodiments, the shape of the one or more valve sleeve ports optimizes an alignment of jet forces of hydraulic fluid flowing through the valve assembly. Brief description of the drawings

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

### BRIEF DESCRIPTION OF THE DRAWINGS

The following drawings are for illustrative purposes only and show non-limiting embodiments. Features from different figures may be combined in several embodiments.

FIG. 1 is a side view of an example of a prosthetic knee containing a hydraulic dampener.

FIG. 1A is a perspective rear view of the example prosthetic knee containing a hydraulic dampener in FIG. 1.

FIG. 2 is a perspective side view of an example of a hydraulic dampener.

FIG. 2A is a cross-sectional side view of the example hydraulic dampener in FIG. 2.

FIG. 2B is a perspective rear view of the example hydraulic dampener in FIG. 2.

FIG. 2C is a rear view of the example hydraulic dampener in FIG. 2.

FIG. 2D is a schematic of a flow circuit of the example hydraulic dampener in FIG. 2.

FIG. 3A is an isolated side view of an example accumulator.

FIG. 3B is an isolated side view of the accumulator in FIG. 3A with its coupling removed to show an example filter.

FIG. 3C is an isolated cross-sectional view of the accumulator in FIG. 3B.

FIG. 3D is an isolated view of the inner surface of the proximal end of a coupler of the accumulator in FIG. 3B.

FIG. 4 is an isolated view of an example valve system.

FIG. 5 is an isolated perspective view of an example valve spool.

FIG. 5A is another isolated perspective view of an example valve spool.

FIG. 5B is an isolated cross-section view of an example valve spool.

FIG. 6A is an isolated side view of an example valve sleeve.

FIG. 6B is an isolated rear view of an example valve sleeve.

FIG. 7 is an isolated view of an example valve assembly.

FIG. 7A is a close-up view of the valve assembly in FIG. 7 in one position.

FIG. 7B is another close-up view of the valve assembly in FIG. 7 in another position.

FIG. 7C is a close-up view of the valve assembly in FIG. 7.

FIGS. 8A and 8B are graphs showing the relationship between valve position and resistance during actuation of a prosthetic knee. FIG. 8A is a graph showing the relationship between valve position and differential pressure at various stages of actuation. FIG. 8B is a graph showing the relationship between valve position and torque forces at various stages of hydraulic cylinder actuation.

FIG. 9A illustrates a perspective rear view of an example prosthetic knee with a rear housing.

FIG. 9B illustrates a perspective rear view of the example prosthetic knee of FIG. 9A, with a panel removed from the rear housing.

FIG. 9C illustrates an exploded perspective rear view of the example prosthetic knee of FIG. 9A showing the rear housing decoupled from a frame of the example prosthetic knee.

### DETAILED DESCRIPTION

The following detailed description is directed to certain specific embodiments of prosthetic devices and methods. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. The embodiments, examples of which are illustrated in the accompanying drawings, are set forth in detail below. Wherever possible, the same reference numbers are used throughout the drawings to refer to the same or like parts.

Microprocessor-controlled hydraulic prosthetic knees are an ideal solution for controlling the rotation of the prosthetic knee joint for above-knee amputees. These systems employ one or more microprocessors and various sensors that detect, respond, and react to the bending of the knee joint by modifying the hydraulic resistance in both flexion and extension directions. Such modification of hydraulic resistance can be achieved by the use of a valve system coupled with a hydraulic cylinder. While the following disclosure describes a hydraulic valve system with respect to a microprocessor-controlled hydraulic prosthetic knee, one of skill in the art will understand that the valve system may be suitable for use in other types of prosthetics (e.g., prosthetic feet, prosthetic ankles, etc.) and prosthetics that are not microprocessor-controlled (e.g., prosthetics where hydraulic resistance is manually adjustable by a user or prosthetist by manually changing a valve position of a hydraulic valve system).

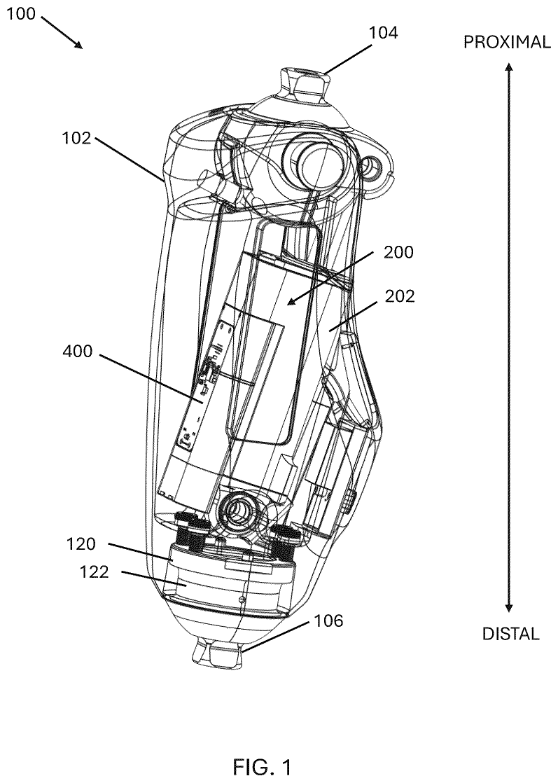

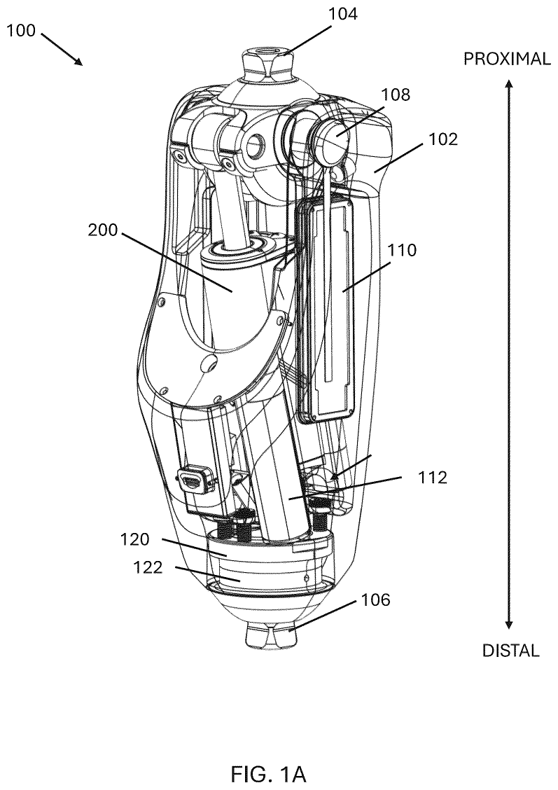

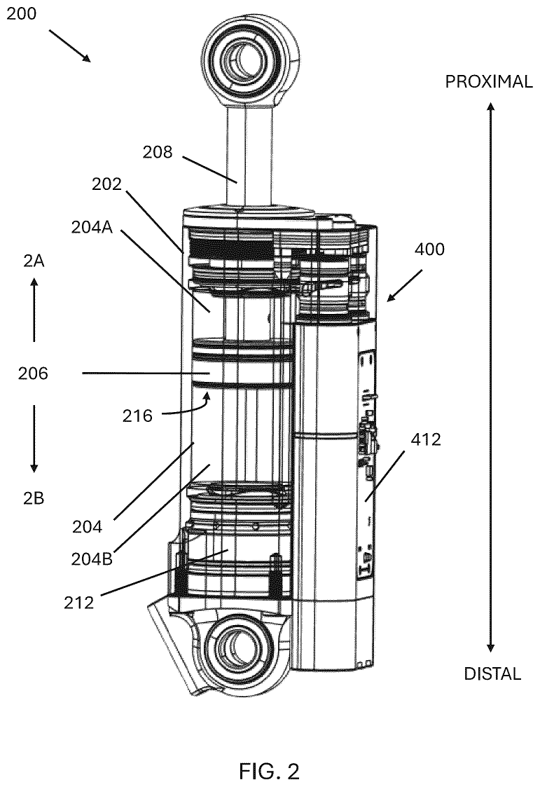

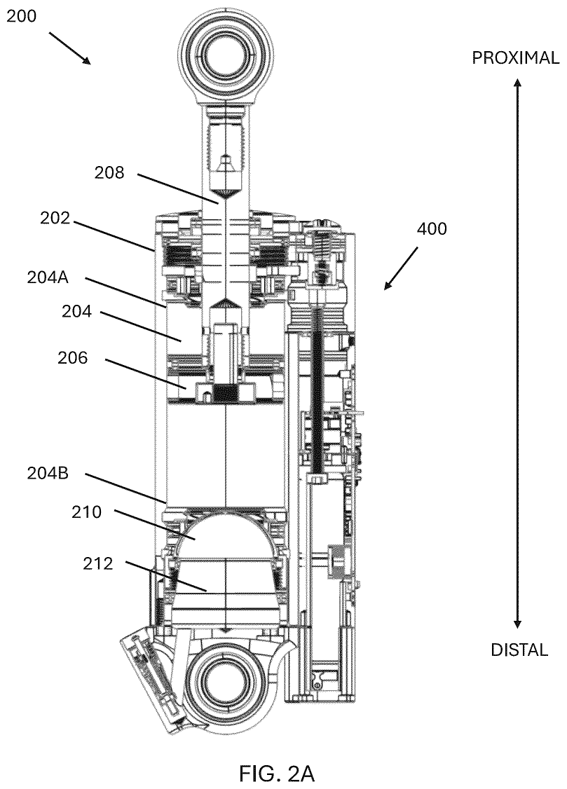

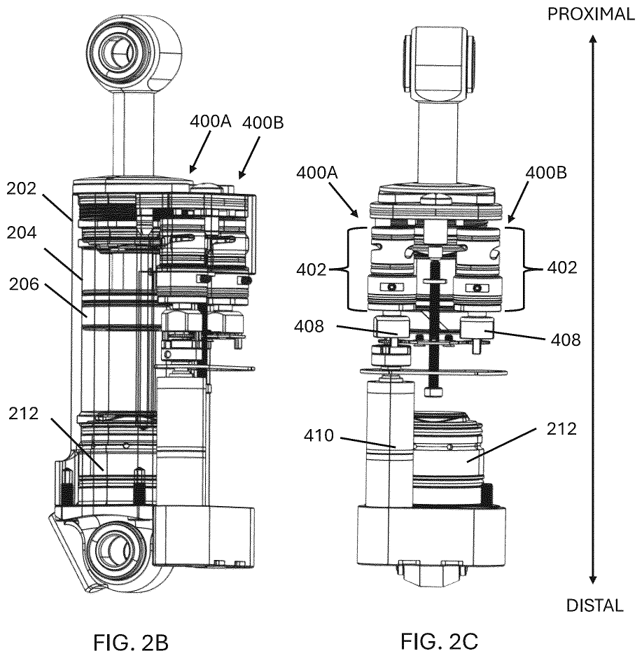

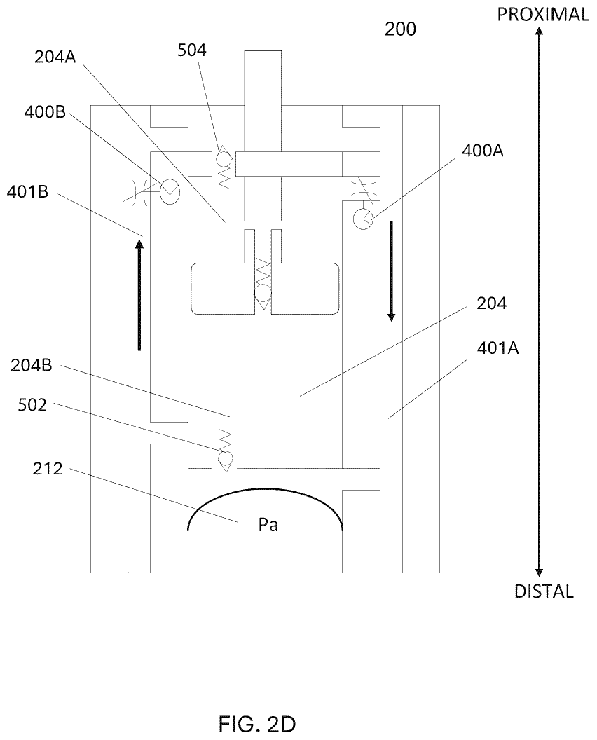

FIGS. 1 and 1A show a side and a perspective rear view, respectively, of an example of a prosthetic knee 100 (e.g. a micro-processor controlled prosthetic knee) containing the hydraulic dampener 200 described herein. The hydraulic dampener 200 includes a hydraulic cylinder 202 and a valve system 400. As described further below with respect to FIG. 2, a piston 206 can move within a chamber 204 of the hydraulic cylinder 202 (e.g., to adjust a position of the prosthetic knee). The prosthetic knee 100 may additionally include one or more sensors 108 and a knee microprocessor 110. The one or more sensors 108 may detect position (e.g., knee angle) and/or motion data of the prosthetic knee 100 during gait and transmit the data to the knee microprocessor 110. The knee microprocessor 110 may then use the position (e.g., knee angle detected by the one or more sensors 108) and/or motion data to control the hydraulic dampener 200 in order to adjust the resistance of the prosthetic knee 100. The knee microprocessor 110, one or more sensors 108, and hydraulic dampener 200 of the prosthetic knee 100 may be powered by a battery 112 and housed within a frame 102 of the prosthetic knee 100. The frame 102 is shown as transparent in FIGS. 1-2 to facilitate illustrating the internal components housed within the frame 102. In some embodiments, there may be additional microprocessors (e.g., separate from the knee microprocessor 110) associated with other components of the prosthetic knee 100. For example, there may be a microprocessor (e.g., the microprocessor 412 shown in FIG. 2)associated with the hydraulic dampener 200 and/or a microprocessor (not shown) associated with the load cell 120, as discussed further below.

The proximal end of the prosthetic knee 100 may include a proximal connector 104 (e.g., a pyramid connector) for connecting the prosthetic knee 100 to an upper leg prosthetic device (e.g., a socket worn by an amputee over their upper leg). The distal end of the prosthetic knee 100 may include a distal connector 106 (e.g., a pyramid connector) for connecting the prosthetic knee to a lower leg prosthetic device (e.g., a pylon that couples to a prosthetic foot, a prosthetic foot, etc.). The distal end of the prosthetic knee 100 may additionally include a load cell 120 that detects and measures loads transmitted through the prosthetic knee 100 during ambulation. The load cell 120 includes a deformable portion 122 containing a transducer assembly (not shown) with one or more strain gauges. The strain gauges measure axial, shear, and bending loads applied to the prosthetic knee 100 based on deformation of the deformable portion 122 during gait cycles. The load cell 120 may include a microprocessor (not shown) that uses the measurements of the one or more strain gauges to adjust the flexion and/or extension resistance of the prosthetic knee 100 in real-time, providing a more natural and stable gait by adapting to different terrains and activities. In some embodiments, there may be one or more (e.g., one, two, three, four, etc.) strain gauges disposed on a first side of the deformable portion 122 and one or more (e.g., one, two, three, four, etc.) strain gauges disposed on a second, opposite side of the deformable portion 122, such that all strain gauges are aligned on a single plane. This configuration results in each of the strain gauges measuring loads applied to the prosthetic knee 100 in a single plane direction, resulting in more accurate readings and a smoother result for the end user. There may be the same number of strain gauges disposed on each opposing side of the deformable portion 122. Alternatively, a first side of the deformable portion 122 may have a greater or lesser number of strain gauges than a second, opposite side of the deformable portion 122.

FIG. 2 is an isolated side view of an example hydraulic dampener 200. FIGS. 2A-2C show cross-sectional, perspective, and rear views of the hydraulic dampener 200, respectively. The hydraulic dampener 200 includes a hydraulic cylinder 202 and one or more valve systems 400. The hydraulic cylinder 202 defines a chamber 204 containing hydraulic fluid. The hydraulic cylinder 202 may be movably coupled to a piston 206 and a piston rod 208. The piston 206 may be positioned at a distal end of the piston rod 208. The piston rod 208 may extend through the top surface of the hydraulic cylinder 202 into the chamber 204 such that the distal end of the piston rod 208, including the piston 206, is contained within the hydraulic cylinder 202. The piston rod 208 and piston 206 may both travel linearly within the chamber 204 (e.g., in the directions indicated by Arrow 2A and Arrow 2B in FIG. 2) during gait. The chamber 204 of the hydraulic cylinder 202 may be in fluid communication with the one or more valve systems 400.

The one or more valve systems 400 include a valve assembly 402, motor coupler 408, and gearmotor 410. As shown in FIGS. 2B and 2C, the hydraulic dampener 200 may include two valve systems 400—a first valve system 400A operated to control resistance in an extension movement of the prosthetic device (e.g., the prosthetic knee 100), and a second valve system 400B operated to control resistance in a flexion movement of the prosthetic device (e.g., the prosthetic knee 100) (note that the gearmotor 410 of the second valve system 400B is excluded in FIGS. 2B and 2C, but is similar to the illustrated gearmotor 410). However, in other examples, the hydraulic dampener 200 may include only one valve system 400 that is operated to control resistance in both flexion and extension movements. The hydraulic dampener 200 may additionally include a microprocessor 412 operable to adjust an amount of resistance within the hydraulic dampener 200 in response to programmed instructions and/or signals detected by one or more sensors 108 of the prosthetic knee 100, etc.

FIG. 2D is a schematic diagram showing an example flow circuit showing how hydraulic fluid flows within the hydraulic dampener 200 during gait. As shown, in some embodiments, hydraulic fluid flows along a channel 401B from the distal end 204B to the proximal end 204A of the chamber 204 through the second (e.g., flexion) valve system 400B during flexion. During extension, hydraulic fluid flows along a channel 401A from the proximal end 204A to the distal end 204B through the first (e.g., extension) valve system 400A. The hydraulic dampener 200 may additionally include a first one-way flow valve 502 through which oil stored in the accumulator 212 (described below) can flow into the distal end 204B of the chamber 204. Additionally, there may be a second one-way flow valve 504 through which hydraulic fluid can flow from the channel 401B into the proximal end 204A of the chamber 204. During flexion, hydraulic fluid flows from the distal end 204B of the chamber 204 along channel 401B to proximal end 204A of the chamber 204 (e.g. via the second one-way flow valve 504) and to the accumulator 212 via channel 401A. The flow of hydraulic fluid during flexion may cause the proximal end 204A of the chamber to fill with hydraulic fluid, and the diaphragm of the accumulator 212 may be compressed. During extension, hydraulic fluid flows from the proximal end 204A of the chamber 204 to the distal end 204B of the chamber 204 along the channel 401A, and hydraulic fluid in the accumulator 212 flows into the distal end 204B of the chamber 204 (e.g., via the first one-way flow valve 502). The flow of hydraulic fluid during extension may cause the distal end 204B of the chamber 204 to fill with hydraulic fluid, and the diaphragm of the accumulator 212 may expand.

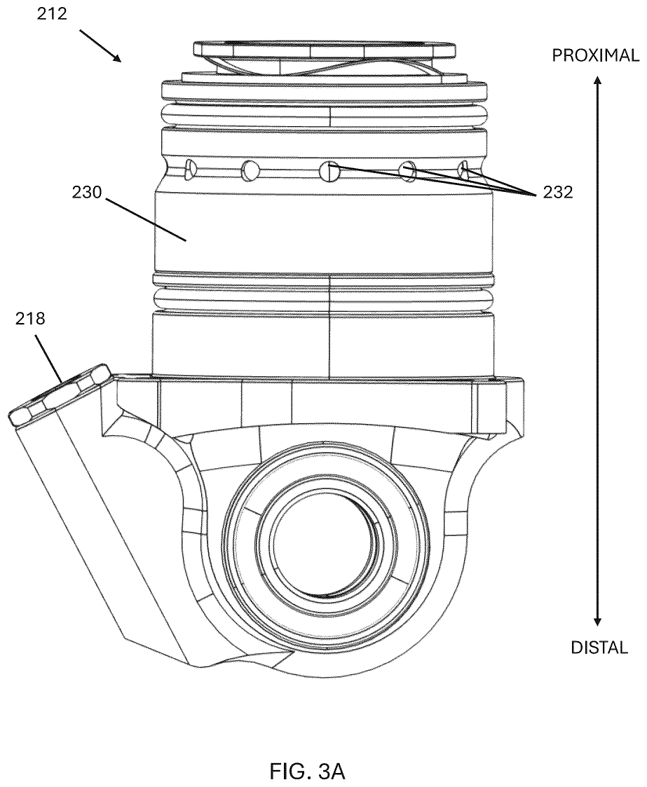

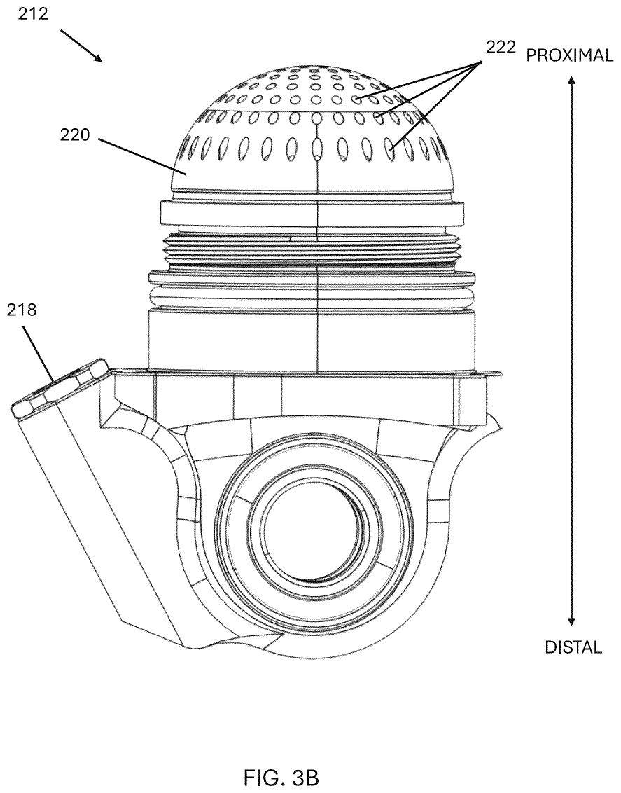

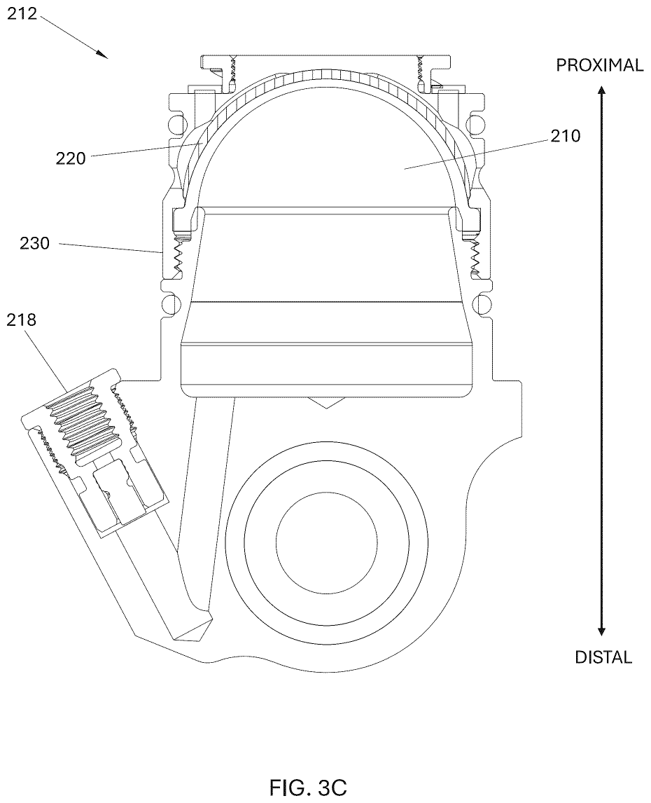

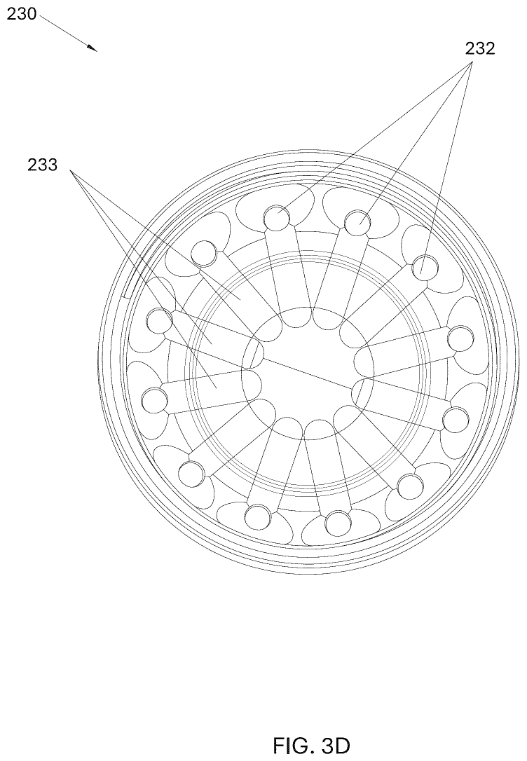

As the prosthetic knee 100 flexes, the piston 206 travels in the direction indicated by Arrow 2B in FIG. 2, displacing the hydraulic fluid contained in the distal end 204B of the chamber 204 and forcing it through the valve system 400 towards the proximal end 204A of the chamber 204. As discussed above, the hydraulic dampener 200 may additionally include an accumulator 212 positioned at the distal end 204B of the chamber 204 to store hydraulic fluid. The accumulator 212 may include a diaphragm 210 as shown in the cross-sectional view of the hydraulic dampener 200 in FIG. 2A, and the cross-sectional view of the accumulator 212 in FIG. 3C. The diaphragm 210 may be made from a compliant, flexible material (e.g. rubber) and have a hemi-spherical or dome shape defining a hollow chamber filled with a gas (e.g., air, nitrogen). As the prosthetic knee 100 approaches a fully flexed state, the piston 206 compresses the diaphragm 210, increasing the gas pressure in the accumulator 212. Additionally, as the diaphragm 210 is compressed, the area in the accumulator 212 in which excess hydraulic fluid (e.g., hydraulic fluid that is not displaced towards the proximal end 204A of the chamber 204) can be stored increases.

As the prosthetic knee 100 extends, the piston 206 travels in the direction indicated by Arrow 2A in FIG. 2, displacing the hydraulic fluid at the proximal end 204A of the chamber, and forcing it through the valve system 400 towards the distal end 204B of the chamber. Motion of the piston 206 in the extension direction may be aided by the increased pressure in the accumulator. Because the piston rod 208 reduces the area in the proximal end 204A of the chamber 204 as compared to the distal end 204B of the chamber 204, there is a greater surface area (e.g., the distal facing surface 216 of the piston 206) at the distal end 204B of the chamber 204 than at the proximal end 204A of the chamber 204 on which the hydraulic fluid can act. When the prosthetic knee 100 transitions from flexion to extension, the increased pressure in the accumulator 212 is released and is transferred to the piston 206 in the extension direction (e.g., arrow 2A in FIG. 2), aiding motion of the piston 206 in the extension direction.

FIGS. 3A-3C show various isolated views of an example accumulator 212. FIG. 3A is an isolated side view of the accumulator 212. The accumulator 212 may include a coupler 230 that allows it to couple to the distal end of the hydraulic dampener 200. The coupler 230 may include a plurality of openings 232 through which hydraulic fluid can flow between the accumulator 212 and the distal end 204B of the chamber 204. As shown in FIG. 3D, the inner surface of the proximal end of the coupler 230 may additionally include a plurality of recessed channels 233 to aid in directing the hydraulic fluid towards the plurality of openings 232. As discussed above, the accumulator 212 may include a diaphragm 210, which may be made from a compliant, flexible material (e.g. rubber) and have a hemi-spherical or dome shape defining a hollow chamber filled with a gas (e.g., air, nitrogen). The accumulator 212 may additionally include a valve 218 through which a gas (e.g., air, nitrogen) can be delivered into the diaphragm 210.

In some embodiments, the accumulator 212 may include a filter 220 (e.g., a spacer with a plurality of apertures or holes) disposed over the diaphragm 210 to aid the flow of hydraulic fluid between the accumulator 212 and the distal end 204B of the chamber 204, as shown in FIG. 3B, which is an isolated, side view of the accumulator 212 with coupler 230 removed. The filter 220 may be made from a rigid material (e.g., plastic) and have a hemi-spherical or dome shape corresponding to the shape of the diaphragm 210 as shown in the cross-sectional view of the accumulator 212 in FIG. 3C. The filter 220 may include a plurality of openings 222 through which hydraulic fluid can flow between the accumulator 212 and the distal end 204B of the chamber 204. The filter 220 may additionally assist the flow of hydraulic fluid by inhibiting (e.g., preventing) the diaphragm 210 from extending into and blocking one or more of the plurality of channels 233 on the inner surface of the proximal end of the chamber 204.

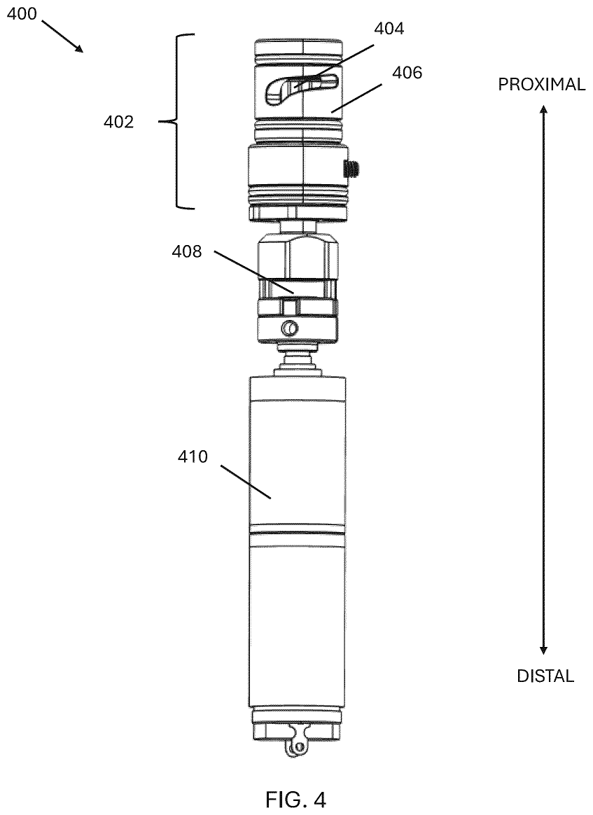

FIG. 4 is an isolated view of an example valve system 400. The valve system 400 may include a valve assembly 402 containing a valve spool 404 surrounded by a valve sleeve 406, a motor coupler 408, and a gearmotor 410. The motor coupler 408 transfers mechanical energy from the gearmotor 410 to the valve spool 404 of the valve assembly 402 to cause the valve spool 404 to rotate within the valve sleeve 406. In some embodiments, the knee microprocessor 110 of the prosthetic knee 100 may control the gearmotor 410 based on position (e.g., knee angle, first derivative of knee angle or knee angle velocity, second derivative of knee angle or knee angle acceleration, etc.) and/or motion data captured by the one or more sensors 108 of the prosthetic knee. Additionally and/or alternatively, the microprocessor (not shown) associated with the load cell 120 may adjust the flexion and/or extension resistance of the prosthetic knee 100 based on sensed load data from the measurements of the one or more strain gauges. Additionally and/or alternatively, the microprocessor 412 of the dampener 200 may adjust hydraulic resistance within the hydraulic dampener 200 (e.g., based on programmed instructions and/or data from one or more sensors 108 of the prosthetic knee 100). Additionally and/or alternatively, the valve system 400 may be controlled manually. As the rotational position of the valve spool 404 changes with respect to the valve sleeve 406, the amount and/or rate of hydraulic fluid flowing through the valve assembly 402 changes, which in turn varies the hydraulic resistance within the hydraulic dampener 200 to control the flexion and extension motion, as discussed further below.

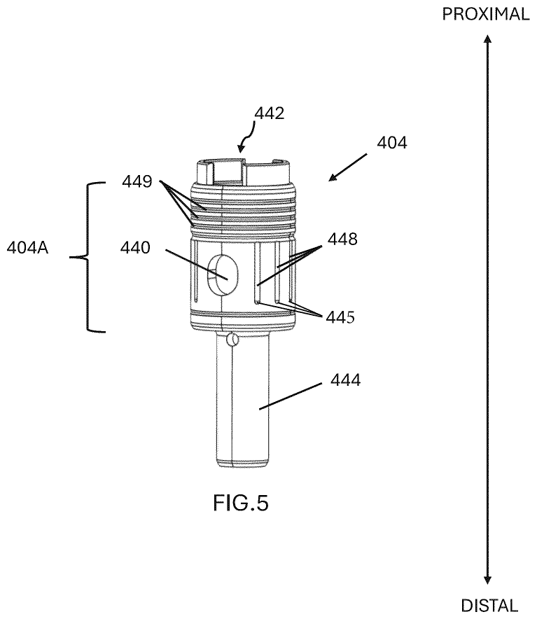

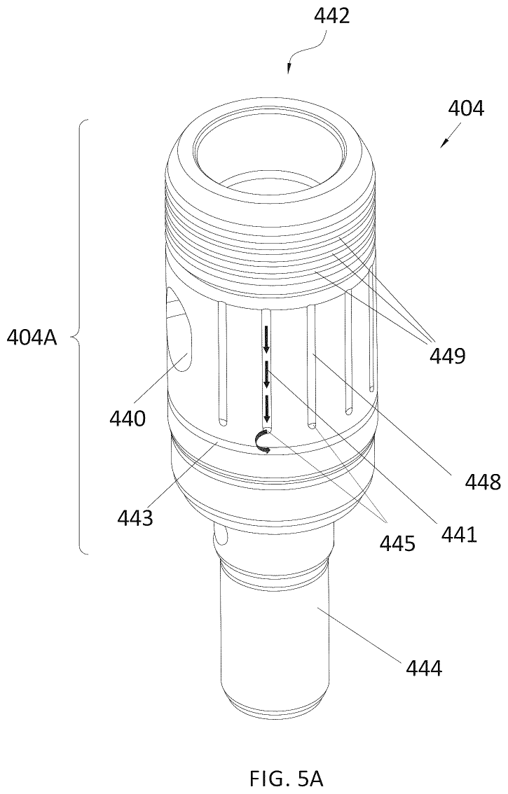

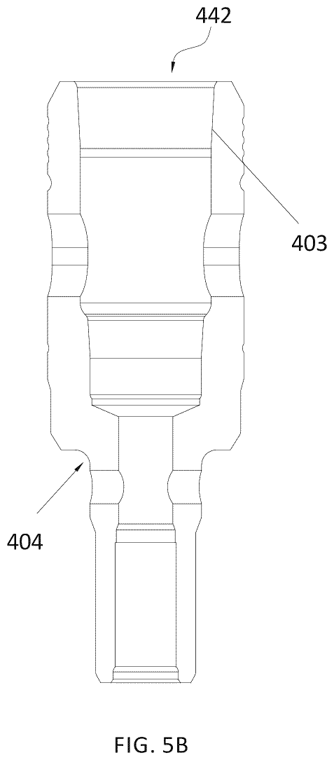

FIGS. 5-5A show an isolated view of an example valve spool 404 of the valve assembly 402. The proximal end of the valve spool 404 may be a hollow cylindrical structure 404A defining an opening 442 to allow for hydraulic fluid to flow into and/or out of the valve assembly 402. The hollow cylindrical structure 404A may have one or more (e.g., one, two, three, four, etc.) openings or ports 440. The one or more ports 440 may be arranged or distributed evenly around the circumference of the hollow cylindrical structure 404A. The valve spool 404 features 180° symmetry around its center of rotation. The one or more valve spool ports 440 may allow for hydraulic fluid flow therethrough (e.g., between the valve spool 404 and the hydraulic cylinder 202 via a hydraulic flow path). The profile of the ports 440 defines a small axial channel in order to reduce jet forces from the hydraulic fluid flow. This in turn allows the valve spool 404 to act similarly to a hydrostatic bearing to reduce friction as the valve spool 404 rotates within the valve sleeve 406, and to minimize cavitation and noise. In some embodiments, each valve spool port 440 may have an ovular shape, although other shapes may also be suitable (e.g., circular, square, etc.).

The proximal end of the valve spool 404 may additionally include one or more (e.g., three, six, eight) recessed linear or axial grooves 448 disposed around the circumference of the hollow cylindrical structure 404A between the one or more ports 440. The distal end of the axial groove(s) 448 may include a curved transition portion 445 that extends between the recessed surface of the axial groove 448 and the surface of the hollow cylindrical structure 404A. FIG. 5A illustrates how hydraulic fluid flows through the valve assembly 402. A small amount of the hydraulic fluid 441 may be captured by the axial grooves 448 and wicked out into the area between the outer surface of the hollow cylindrical structure 404A of the valve spool 404 and the inner surface of the valve sleeve 406 by the curved transition portion 445, and into a radial groove 443 positioned distal to the axial grooves 448. This advantageously reduces friction during rotation of the valve spool 404 within the valve sleeve 406. The axial grooves 448 additionally inhibit (e.g., prevent) hydrostatic force imbalance, and maintain the valve spool 404 centered within the valve sleeve 406 during the rotation of the valve spool 404 within the valve sleeve 406 when throttling flows at high pressures. While the axial grooves 448 are shown to be parallel relative to a central axis of the valve spool 404, the axial grooves 448 may alternatively be angled with respect to the central axis of the valve spool 404.

The proximal end of the valve spool 404 may additionally include one or more (e.g., three, six, eight) recessed radial or circumferential grooves 449. The recessed radial or circumferential grooves 449 may additionally aid in reducing hydrostatic force imbalance during periods of high pressure fluid flow. The combination of the axial grooves 448 and radial circumferential grooves 449 may significantly reduce torque requirements and inhibit (e.g., prevent) hydrostatic force imbalance when the valve assembly 402 is near fully closed. As hydraulic fluid flows through the valve assembly 402, a small amount of the hydraulic fluid may be captured by the axial grooves 448 and/or recessed radial or circumferential grooves 449, generating a thin film of hydraulic fluid around the solid portions of the hollow cylindrical structure 404A, reducing friction during rotation of the valve spool 404 within the valve sleeve 406. Advantageously, this significantly increases the ability of the valve spool 404 to rotate within the valve sleeve 406, providing a smoother experience to the end user.

The recessed axial grooves 448 and the recessed radial or circumferential grooves 449 of the valve spool 404 provide a cost-effective alternative to traditional hydrostatic bearings. Manufacturing traditional hydrostatic bearings generally involves machining complex pockets and orifices into a valve body, while the axial grooves 448 and recessed radial or circumferential grooves 449 have much simpler geometries. Manufacturing the valve spool 404 may involve first, boring the opening 442 and then machining the axial grooves 448 on a lathe in a single setup. The outer diameter of the hollow cylindrical structure 404A may then be ground down on a fixture with very low runout to ensure that each of the axial grooves 448 has a uniform depth. The inner diameter of the hollow cylindrical structure 404A (e.g., the inner diameter of the opening 442) may have a tapered profile 403, as shown in FIG. 5B, to aid in minimizing runout during the grinding down of the hollow cylindrical structure 404A.

The distal end of the valve spool 404 may include a valve post 444. The distal end of the valve post 444 may be operably coupled to the proximal end of the motor coupler 446. Rotational motion is transferred from the gearmotor 410 by the motor coupler 446 to the valve spool 404 via the valve post 444, causing rotation of the valve spool 404 within the valve sleeve 406.

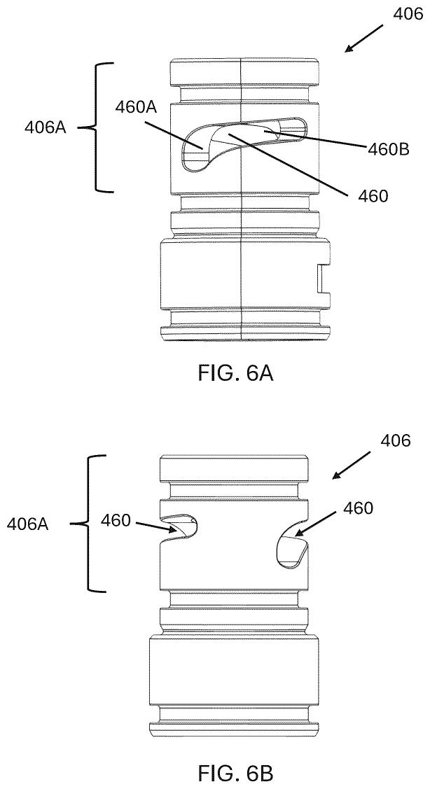

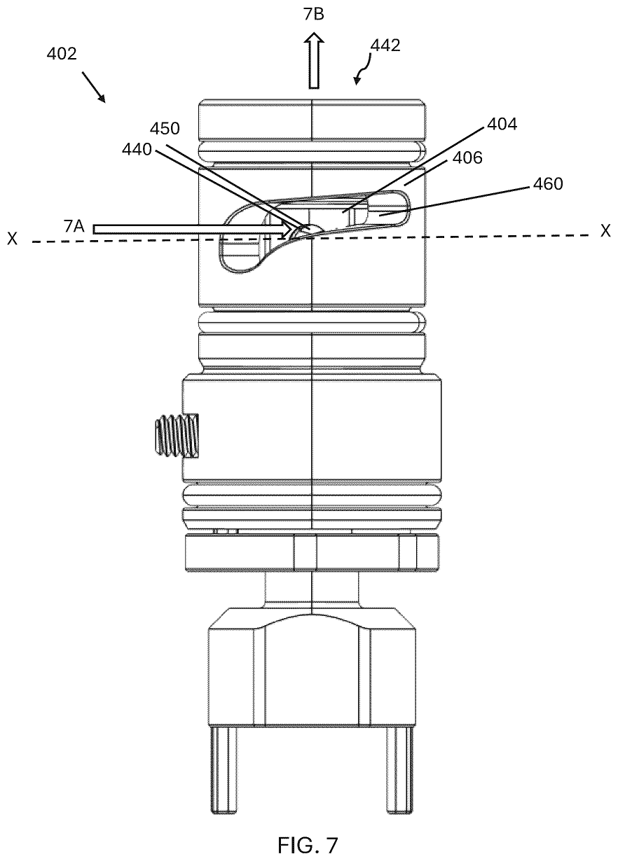

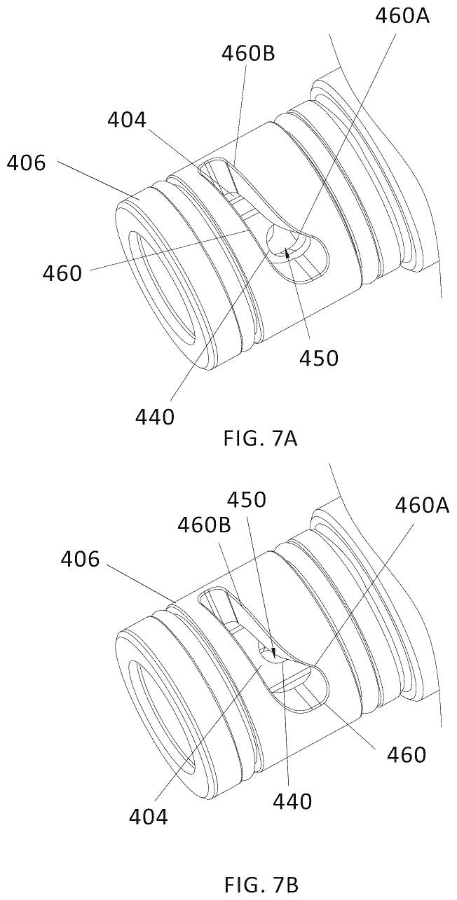

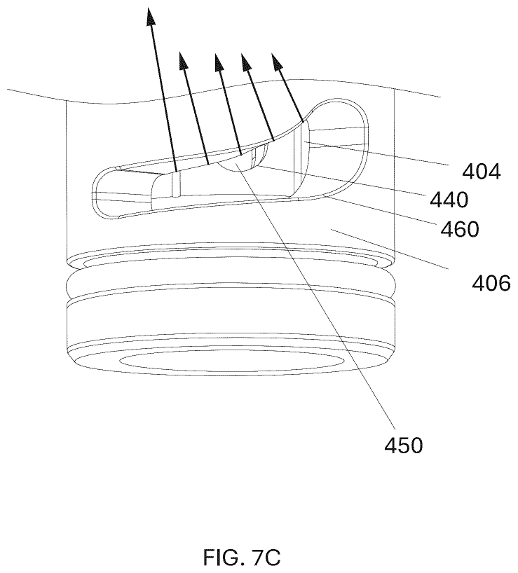

FIGS. 6A and 6B are isolated side and rear views, respectively, of the example valve sleeve 406. The proximal end of the valve sleeve 406 may be a hollow cylindrical structure 406A similar to the cylindrical structure 404A of the proximal end of the valve spool 404, but the circumference of the cylindrical structure 406A of the valve sleeve 406 may be larger than the circumference of the cylindrical structure 404A of the valve spool 404 such that the valve spool 404 may fit inside of the valve sleeve 406 as discussed above with respect to FIGS. 5-5A. The proximal end of valve sleeve 406 may include one or more (e.g., one, two, three, four, etc.) openings or ports 460 (e.g., distributed evenly) around the circumference of the cylindrical structure 406A. In some embodiments, the one or more ports 460 may be distributed or arranged evenly around the circumference of the cylindrical structure 406A such that the valve sleeve 406 features 180° symmetry. However, in other embodiments, the one or more ports 460 may be distributed in another manner such that valve sleeve 406 may not feature 180° symmetry. The number of ports 460 in the valve sleeve 406 can be equal to the number of ports 440 in the valve spool 404. For example, as shown in FIG. 6B, the valve sleeve 406 may include two ports 460 disposed on opposite sides of the cylindrical structure 406A. The one or more ports 460 in the valve sleeve 406 may have a curved shape with an oblong portion 460A and an elongate portion 460B. In some embodiments, the cut or profile of the port 460 may be defined by the equation y(θ)=cn^{θ}m^{θ}, where c, n, and m are tunable constants that may be selected such that the opening 450 formed by the overlapping of the curved port 460 and the valve spool ports 440 increases exponentially as the valve spool 404 rotates, as discussed further below. The curved port 460 may have a larger height at the oblong portion 460A and transition into a smaller height at the elongate portion 460B. The elongate portion 460B may extend at an angle (e.g., 2° or 3°) relative to a horizontal plane or line (e.g., see line X-X in FIG. 7) of the valve assembly 402. The horizontal plane may be transverse or perpendicular to a central axis defined by the valve assembly. The characteristic cut of the curved port 460 advantageously optimizes jet force alignment as hydraulic fluid enters the valve assembly 402. Namely, the characteristic cut of the curved port 460 causes the orientation of the hydraulic jet forces to act increasingly axially (i.e., as opposed to radially) as the valve assembly 402 closes (e.g. moves from position shown in FIG. 7A to position in FIG. 7B and past said position) and throttling is increased, as shown in FIG. 7C, significantly reducing torque and power requirements of the gearmotor 410 to rotate the valve spool 404 of the valve assembly 402.

The manner in which the profile of the curved port 460 overlaps with the ports 440 of the valve spool 404 during operation of the valve system 400 causes the area through which hydraulic fluid flows into the valve assembly 402 to increase exponentially as the valve spool 404 rotates within the valve sleeve 406, providing improved resolution across all combinations of flow and pressure. This, in turn, yields an approximately exponential relationship between the valve position (e.g., rotation of the valve spool 404 relative to the valve sleeve 406) and a hydraulic resistance provided by the valve system 400. In other examples, the port 460 can have other suitable shapes that yield an approximately exponential relationship between the valve position and the hydraulic resistance provided by the valve system 400.

FIG. 7 shows the example valve assembly 402 containing the valve spool 404 and the valve sleeve 406. As shown, the curved ports 460 may be (at least partially) disposed on the same lateral plane X-X as the valve spool ports 440 when the valve spool 404 is disposed within the valve sleeve 406. The amount and/or rate of hydraulic fluid flow between the valve assembly 402 and the hydraulic cylinder 202 may be controlled by the rotational position of the valve spool 404 relative to the valve sleeve 406. When the valve spool 404 is rotated (e.g., manually, or electronically via the gearmotor 410 and the motor coupler 408) to a position where the valve spool ports 440 are aligned with the valve sleeve ports 460, an opening 450 is formed by the aligned ports 440, 460 that allows hydraulic fluid flow into and out of the valve assembly 402 (e.g., allows hydraulic fluid to flow into the valve assembly 402 via the opening 450 in the direction indicated by Arrow 7A and out of the valve assembly 402 through the proximal end of the valve spool 404 in the direction of Arrow 7B). When the valve spool 404 is rotated (e.g., manually, or electronically via the gearmotor 410 and the motor coupler 408) to a position where the valve spool ports 440 are not aligned with the curved port 460 of the valve sleeve 406, the wall (e.g., solid portion) of the valve sleeve 406 will slow or inhibit (e.g., prevent) hydraulic fluid from flowing into and out of the valve assembly 402, thereby increasing the hydraulic resistance of the dampener 200.

As discussed above, the geometry of the curved ports 460 of the valve sleeve 406 relative to the ovular valve spool ports 440 (e.g., and the overlap between curved port 460 and the valve spool ports 440 as the valve spool 404 is rotated relative to the valve sleeve 406) advantageously provides or results in an approximately exponential relationship between the hydraulic resistance and the rotational position of the valve spool 404 relative to the valve sleeve 406 (e.g., a rotational position of the valve assembly 402). When a valve spool port 440 is in line with the oblong portion 460A of the curved port 460, as shown in FIG. 7A, more hydraulic fluid may flow into and out of the valve assembly 402 through the opening 450 and the hydraulic dampener 200 can have a relatively lower hydraulic resistance. As the valve spool 404 is rotated and passes relative to the elongate portion 460B of the curved port 460, as shown in FIG. 7B, the amount of hydraulic fluid flow through the valve assembly 402 (e.g., the amount that passes through the opening 450 formed by the overlapping portions of the valve spool port 440 and the curved port 460 of the valve sleeve 406) is gradually decreased until the valve spool port 440 is no longer aligned with the curved port 460 (e.g., the valve spool port 440 is no longer accessible via the curved port 460), at which point hydraulic fluid flow is blocked by the solid portion of the valve sleeve 406. The valve sleeve 406 may further include one or more seals (e.g., O-rings) to inhibit (e.g., prevent) unwanted hydraulic fluid flow around the valve assembly 402 (e.g., so that hydraulic fluid only passes through the valve assembly 402 via the opening 450 formed by the overlap of the valve spool ports 440 and curved ports 460). Having an exponential relationship between valve rotation and hydraulic resistance advantageously allows for more precise control over the change in resistance of the prosthetic joint (e.g., prosthetic knee 100) as the valve spool 404 is rotated relative to the valve sleeve 406 (e.g., to change the valve position).

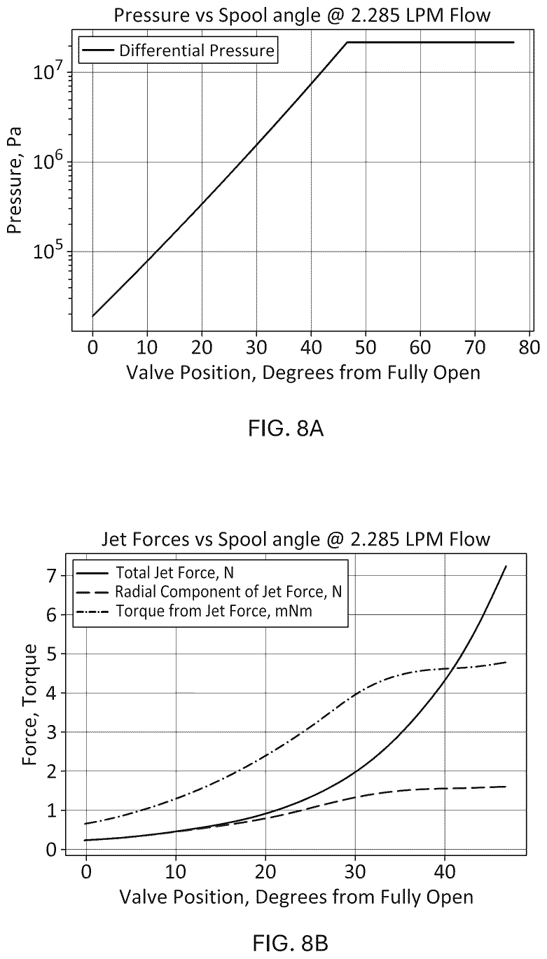

FIGS. 8A and 8B are graphs showing the relationship between valve position and resistance during actuation of the prosthetic knee 100. FIG. 8A is a log-linear graph showing the relationship between valve position and differential pressure at various stages of actuation. As shown, differential pressure increases exponentially as valve spool 404 rotates within the valve sleeve 406 until the valve assembly 402 closes (e.g., when the opening 450 is aligned with a wall of the valve sleeve 406). FIG. 8B is a graph showing the relationship between valve position and torque forces at various stages of hydraulic cylinder actuation. As shown, the total jet force of hydraulic fluid increases exponentially until the valve assembly 402 closes (e.g., when the opening 450 is aligned with a wall of the valve sleeve 406). As discussed above, the characteristic cut of the curved port 460 causes the orientation of the jet forces to act increasingly axially (i.e., as opposed to radially) as the valve assembly 402 closes and throttling is increased. The axial jet forces assist in closing the valve assembly 402, significantly reducing torque and power requirements of the gearmotor 410.

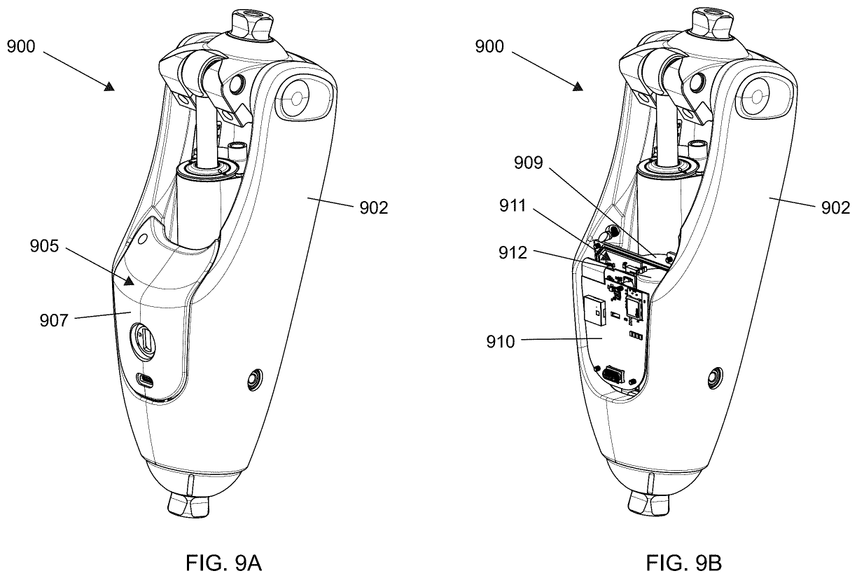

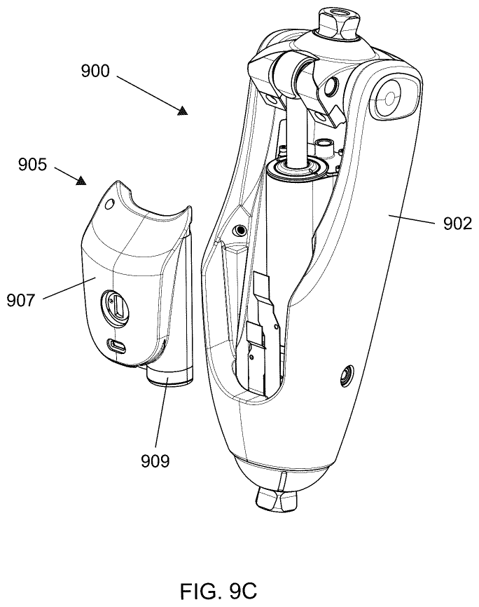

FIGS. 9A-9C show a perspective rear view of another example of a prosthetic knee 900. The prosthetic knee 900 may include any of the features of the prosthetic knee 100, and the prosthetic knee 100 may include any of the features of the prosthetic knee 100.

The prosthetic knee 900 includes a rear housing 905 coupled to the frame 102. The knee microprocessor 910 and/or the battery 912 are positioned in an inner volume 911 of the rear housing 905. The rear housing 905 may be removably coupled to the frame 902 so the rear housing 905 can be removed from the rest of the prosthetic knee 900, as shown in FIG. 9C.

Various modifications to the embodiments described in this disclosure will be readily apparent to those skilled in the art, and the generic principles defined herein may be applied to other embodiments without departing from the spirit or scope of this disclosure. Thus, the disclosure is not intended to be limited to the embodiments discussed herein but is to be accorded the widest scope consistent with the claims, the principles and the novel features disclosed herein. The word “example” is used exclusively herein to mean “serving as an example, instance, or illustration.” Any embodiment described herein as “example” is not necessarily to be construed as preferred or advantageous over other embodiments, unless otherwise stated.

Certain features that are described in this specification in the context of separate embodiments also may be embodied in combination in a single embodiment. Conversely, various features that are described in the context of a single embodiment also may be embodied in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the order shown or in sequential order, or that all illustrated operations be performed, to achieve desirable results. Additionally, other embodiments are within the scope of the following claims. In some cases, the actions recited in the claims may be performed in a different order and still achieve desirable results.

Conditional language, such as “may,” “could,” “might,” “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A hydraulic dampener for a prosthetic device, comprising:
a hydraulic cylinder; and
a valve system in fluid communication with the hydraulic cylinder, the valve system operable to allow flow of a hydraulic fluid to and from the hydraulic cylinder, the valve system comprising:
a valve assembly comprising:
a valve spool having one or more valve spool ports and a plurality of recessed axial grooves; and
a valve sleeve surrounding the valve spool so that the valve spool is rotatable relative to the valve sleeve, the valve sleeve comprising one or more valve sleeve ports,
wherein a hydraulic resistance of the valve assembly varies approximately exponentially with a change in valve position of the valve assembly caused by rotating the valve spool relative to the valve sleeve to vary an alignment between the one or more valve spool ports and the one or more valve sleeve ports.

2. The hydraulic dampener of claim 1, wherein the valve spool comprises a first cylindrical body, and a valve post, the valve post extending distally from the first cylindrical body, the one or more valve spool ports arranged circumferentially around the first cylindrical body.

3. The hydraulic dampener of claim 1, wherein the one or more valve sleeve ports is a curved valve sleeve port comprising an oblong portion and an elongate portion.

4. The hydraulic dampener of claim 1, wherein the valve sleeve comprises a second cylindrical body, the one or more valve sleeve ports arranged circumferentially around the second cylindrical body.

5. The hydraulic dampener of claim 4, wherein the valve sleeve further comprises one or more axial grooves disposed on the second cylindrical body between the one or more valve sleeve ports.

6. The hydraulic dampener of claim 4, wherein each of the one or more valve spool ports defines a small axial channel through which hydraulic fluid can flow through the valve assembly.

7. A hydraulic valve assembly comprising:
a valve spool having one or more valve spool ports and a plurality of recessed axial grooves; and
a valve sleeve surrounding the valve spool so that the valve spool is rotatable relative to the valve sleeve, the valve sleeve comprising one or more valve sleeve ports,
wherein a hydraulic resistance of the valve assembly varies approximately exponentially with a change in valve position of the valve assembly caused by rotating the valve spool relative to the valve sleeve to vary an alignment between the one or more valve spool ports and one or more valve sleeve ports.

8. The hydraulic valve assembly of claim 7, wherein the one or more valve sleeve ports is a curved valve sleeve port comprising an oblong portion and an elongate portion.

9. The hydraulic valve assembly of claim 8, wherein the elongate portion extends at an angle relative to a plane transverse to an axis of the valve assembly.

10. The hydraulic valve assembly of claim 9, wherein the plane is perpendicular the axis of the valve assembly.

11. The hydraulic valve assembly of claim 7, wherein each of the one or more valve spool ports defines a small axial channel through which hydraulic fluid can flow through the hydraulic valve assembly.

12. The hydraulic valve assembly of claim 11, wherein the one or more valve spool ports are configured to at least partially overlap with the one or more valve sleeve ports.

13. The hydraulic valve assembly of claim 7, wherein the valve spool further comprises one or more radial grooves disposed around a circumference of the valve spool.

14. A device for actuation of a prosthetic device, comprising:
a hydraulic cylinder;
a piston movably coupled to the hydraulic cylinder and configured to linearly move within the hydraulic cylinder; and
a motorized valve system, the motorized valve system comprising:
a motor, and
a valve assembly, comprising:
a valve spool having one or more valve spool ports and a plurality of recessed axial grooves; and
a valve sleeve surrounding the valve spool so that the valve spool is rotatable relative to the valve sleeve, the valve sleeve comprising one or more valve sleeve ports,
wherein a hydraulic resistance of the valve assembly varies approximately exponentially with a change in valve position of the valve assembly caused by rotating the valve spool relative to the valve sleeve to vary an alignment between the one or more valve spool ports and one or more valve sleeve ports.

15. The device of claim 14, wherein the valve spool comprises a first cylindrical body, and a valve post, the valve post extending distally from the first cylindrical body, the one or more valve spool ports arranged circumferentially around the first cylindrical body.

16. The device of claim 15, wherein the valve spool further comprises one or more axial channels disposed on the first cylindrical body between the one or more valve spool ports.

17. The device of claim 14, wherein the valve sleeve comprises a second cylindrical body, the one or more valve sleeve ports arranged circumferentially around the second cylindrical body.

18. The device of claim 14, wherein the one or more valve sleeve ports are curved valve sleeve ports having an oblong portion with a first height, and an elongate portion with a second height, and wherein the first height is greater than the second height.

19. The device of claim 14, further comprising a coupler configured to operably couple the motor to a distal end of the valve assembly, wherein actuation of the motor causes a rotation of the valve spool within the valve sleeve.

20. The device of claim 14, wherein the prosthetic device is a prosthetic knee.
